(19) United States
(12) Patent Application Publication
(10) Pub. No.: US 2017/0196808 A1
(43) Pub. Date: Jul. 13, 2017

(54) METHOD OF OBTAINING A PHARMACOLOGICALLY ACTIVE LIPOSOMAL QUERCETIN-CONTAINING PRODUCT

(71) Applicant: TOVARISTVO Z OBMEZHENOU VIDPOVIDALNISTU 'NANOMEDTRAST', Kyiv (UA)

(72) Inventors: Ganna Savivna GRYGOR'IEVA, Kyiv (UA); Iuriy Myhaylovych KRASNOPOL'SKYY, Kharkiv (UA); Nataliia Philimonivna KONAJOVYCH, Vasyl'Kyivs'Ka obl. (UA); Nataliia Volodymyrivna PASECHNIKOVA, Kyiv (UA)

(73) Assignee: TOVARISTVO Z OBMEZHENOU VIDPOVIDALNISTU 'NANOMEDTRAST', Kyiv (UA)

(21) Appl. No.: 15/324,009

(22) PCT Filed: Jul. 8, 2015
(86) PCT No.: PCT/UA2015/000059

§ 371 Date: Jan. 5, 2017

(30) Foreign Application Priority Data
Jul. 8, 2014 (UA) A 2014 07695

(51) Int. Cl. A61K 9/127; A61K 31/353

(52) U.S. Cl. CPC A61K 9/127; A61K 31/353

(57) ABSTRACT

The invention discloses a new method of obtaining a pharmacologically active product that is a liposomal composition of phospholipid phosphatidylcholine and physiologically active substance—quercetin (3,3′, 4′, 5, 7-pentaoxyflavone). The substance of the invention to provide a method of optimized parameters sequential processes of dissolution, emulsification, dispersion and lyophilization that results in liposomal quercetin product with proven independent methods liposomal organisation, high stability and pharmacological activity. The high quality of the target product produced by the method claimed ensures the benefits of level and dynamics of integral pharmacological effect in non-clinical study, as compared with liposomal quercetin product produced by the the prototype method. The polytropic pharmacotherapeutic activity of the target product with demonstrated high level of harmlessness by different routes of administration, was to prove in the model of subtotal myocardial ischemia and reperfusion isolated heart performance in Guinea pigs by Langendorff method (the antiarrhythmic effect, normalisation of functional and hemodynamic characteristics of the myocardium), and also at the traumatic keratitis (reparative and anti-inflammatory effect). The invention is intended for use in pharmacy and medicine as a way to obtain a competitive product pharmacotherapy ophthalmologic and cardiologic diseases, adequate to different routes of administration.

The invention relates to pharmaceutics and discloses a method of obtaining a pharmacologically active liposomal quercetin-containing product that features a multifunctional pharmacological profile and may be used for pharmacotherapy, in particular, in cardiology and ophthalmology.

Quercetin (QC), 3,3′, 4′, 5, 7-pentaoxyflavone, belongs to flavonols, to a subclass of flavonoid compounds. QC demonstrates exceptionally high antioxidant activity, which determines its unique multifunctional pharmacotherapeutic properties: anti-inflammatory, antitumor, antispasmodic, antifibrinolytic, antimicrobial actions, etc. [1, 2]. A wide range of therapeutic properties makes QC almost an ideal component of medicinal products. Development of such products is however limited due to extremely low solubility of QC in aqueous media. This explains why the range of known quercetin products has been limited for a long while to oral products only which feature low bioavailability of the active substance.

At the same time, a tempting prospect of the widest clinical use of polytropic properties of QC drove the interest to develop methods to produce QC-containing pharmacological agents which may be suitable for parenteral or other routes of administration.

Known methods of obtaining QC products include an operation of transforming quercetin into soluble state, in the presence of polyvinylpyrolidone [3], mixtures thereof with sodium tetraborate and Trilon B [4,5] or producing an aqueous QC suspension stabilised with preservatives and food acidulants, flavours, dyes [6]. The main disadvantage of these methods is that reproduction thereof requires presence of a large number of non-physiological excipients and an operation of thermal sterilisation [4], which affects harmlessness and stability of target products and suitability of the products for various or combined routes of administration.

Methods which ensure a liposomal organisation of a target product [7] meet, to a large extent, present-day criteria of creating parenteral products based on slightly soluble pharmacologically active substances.

Methods incorporating QC into liposomes using phospholipids—natural lipid matrix components in cell membranes—are known in the prior art. The prior art discloses methods of obtaining liposomal QC products based on liposomes, derived from phosphatidylcholine (PC) or its composition with phosphatidylethanolamine or phosphatidylethanolamine distearate in the presence of different variations of additives, such as cholesterol, hydroxypropyl cyclodextrin, polyethylene glycol, including modified distearyl phosphatidylethanolamine, stearic acid or glycerol monostearate [8-12]. The prior art also discloses a method of obtaining QC-containing nanopowder which uses a so-called “surfactant” (a surrogate of a classic surfactant—a natural complex of surface-active lipoproteid substances) i.e. PC variations with poloxamer, Tween 80, methyl cellulose, sodium deoxycholate, polyethylenised castor oil, etc. [13].

Although operations implementing these methods (direct dispersion of a mixture of components or ultrasound hydration of a lipid film in an aqueous medium or application of an emulsifier etc.) vary by complexity and duration, the general disadvantage is the need to use a number of mainly non-physiological additives, instability and heterogeneity of the size and structure of particles of the QC product so produced, including lack of liposomal organisation of the product. These circumstances complicate parenteral administration and may contribute to undesirable physiological effects [14]. This may explain why target QC products, when the above methods are implemented, may not be positioned as medicinal products with proven, as required by regulatory authorities, high efficacy, harmlessness and pharmaceutical quality.

A method of obtaining a liposomal QC-containing product based on phosphatidylcholine only is known in the prior art [15]. This method produces a target product, which pharmaceutical and pharmacotherapeutic quality along with technological production operations allow promoting the product as a medicinal one. [16] This method is chosen as a prototype of the method claimed since it is the closest analogue based on the set of features, such as: the nature of main components involved in implementation of the method, the essence and the sequence of basic operations and the nature and liposomal organisation of the target product containing QC and having pharmacotherapeutic activity.

The prototype method comprises producing a solution of a mixture of phosphatidylcholine (PC) and quercetin (QC) in ethyl alcohol at a PC to QC ratio (mass fraction) of 1: (0.01-0.10), drying the mixture in vacuum, emulsifying the mixture in an aqueous medium, dispersing the emulsion followed by addition of lactose in the form of an aqueous solution, sequential filtration with smaller pore size filters, sterilisation filtration followed by dispensing and freeze-drying.

The prototype method however requires compliance with certain conditions and completion of operations which may affect method reproducibility and the quality of the target product.

First, in the prototype method, QC is dissolved at a higher temperature, and PC is added to this heated solution, leading to oxidation of substances.

Second, a dried mixture is emulsified in water, the medium temperature is not fixed and pH value is not stabilised, this does not generally promote conditions for phospholipid phase transfer and may provoke uneven structure and charge distribution in lipid particles in the emulsion.

Third, dispersing the emulsion at a fixed pressure of 60 MPa (about 590 atm), as required by the prototype method, prolongs the operation and then increases the size and enhances heterogeneity of liposome size. The latter is very important in terms of intended parenteral administration of the target product.

Fourth, the prototype method uses a rather narrow range of quercetin to lactose ratio (weight fraction), specifically (1-24):(1-30), and the entire amount of lactose is introduced at once at the dispersion stage. In aggregate, these factors may affect a lyophilisation operation, since lactose functions as a cryoprotectant, and stability of the target product in a dosage form suitable for pharmacotherapeutic use.

These circumstances reduce the efficacy of the prototype method in terms of the implementation process and the quality and stability of the target product—a pharmacologically active liposomal quercetin-containing product.

The object of the invention claimed is to develop a method of obtaining liposomal quercetin product, having optimised parameters of operations, which ensures higher quality of the target product, adequate to different routes of administration, in terms of pharmacological activity and stability.

This problem is solved when in the method of obtaining a liposomal quercetin-containing product, including producing a mixture of solutions of phosphatidylcholine and quercetin in ethyl alcohol, drying the mixture in vacuum, emulsifying the mixture in an aqueous medium, dispersing the emulsion, stage-by-stage filtering, sterilisation filtration and freeze-drying, according to the invention, when a mixture of solutions is produced, quercetin is pre-dissolved at a room temperature, the mixture is emulsified at 37-42 oC and lactose solution in phosphate buffer, pH (6.8-7.1), containing 70-90% of total lactose, is used as an aqueous medium, dispersing is subject to stage-by-stage pressure increase from 300 atm to 1,200 atm the emulsion is controlled for particle size, and, after dispersing, lactose solution in phosphate buffer, pH (6.8-7.1), containing 30-10% of total lactose is further added to the emulsion and quercetin to lactose mass ratio is between (1:31) and (1:80).

The following examples illustrate how the method claimed may be implemented and the prototype method is used for comparison.

### Example 1

The method claimed. Dissolve accurately weighed 1.25 g of QC, recalculated on 100% substance (e.g. [17-19]) in 125 mL of ethyl alcohol at a room temperature under stirring. Dissolve accurately weighed 42.0 g of PC (recalculated on 100% substance, e.g., [20-21]) in 150 mL of ethyl alcohol at a room temperature and add to the above QC solution. Mix the solution mixture for 5-7 min, transfer the mixture to a rotary evaporator, and allow alcohol to evaporate completely in vacuum at (40-42) ° C. until a film is formed. When the drying process is over, blow inert gas into the evaporator flask for 20-25 minutes.

Quantitatively remove the film so formed from walls of the evaporator flask at (37-42) oC using 1.45 L of lactose solution (milk sugar, pharmacopoeia grade) in phosphate buffer solution, pH (6.7-7.1) [22], containing 50.0 g of lactose, and shake at 130-140 rpm (e.g. IKA, Germany) and stir for 5-10 minutes until the emulsion becomes homogeneous.

Transfer the emulsion into a reactor of a high pressure homogenizer (e.g., M 110P Microfluidizer Processor, Microfluidics) and allow to disperse at (38-43) ° C. subject to gradual pressure increase from 300 atm to 600 atm and then—to 900 atm for 1-3, 4-8 and 9-10 cycles, respectively. At the dispersion stage, control the emulsion for particle size (e.g. Malvern Zetasizer Nano 5), which, at the end of the process, may not exceed 180 nm.

When homogenisation is over, add 0.05 L of lactose solution in the buffer solution, pH (6.7-7.1), containing 12.5 g of lactose, and mix. Filter the resulting emulsion sequentially through Millipore using 0.8 μm, 0.45 μm and 0.22 μm membranes followed by sterilisation filtration and dispense in glass vials under aseptic conditions.

Allow vials with the emulsion to blast-air freeze efficiently and freeze-dry (e.g. Martin Christ-2-6-D, USA). After drying, blow vials with a lyophilised product with inert gas, close and seal under aseptic conditions.

In Examples 2-6, operations and measures in the method claimed are done as described for Example 1. Changes are described in Table 1.

The target product is light amorphous substance having light-yellow colour with a touch of lemon and a characteristic odour.

### Example 7

The prototype method according to [10]. Dissolve accurately weighed 24 g of QC (e.g. [12, 13]) in 2 L of ethyl alcohol at 50 oC and add 800 g of PC (e.g. [15]) in 2.0 L of ethyl alcohol to the solution. Mix the solution mixture carefully and transfer to a round-bottom flask to be placed on a rotary evaporator, and allow alcohol to completely evaporate in vacuum at 42 oC. When the drying process is over, blow inert gas through the flask for 5 minutes.

Quantitatively remove the lipid film so formed from walls of the flask using approximately 3 L of water for injection and transfer to a glass flask. Once the film is completely removed to the glass flask, add water for injection to make up the volume to 15 L of liquid, stir and mix for 2-3 hours until the emulsion becomes homogeneous.

Transfer the emulsion into a reactor of a high pressure homogenizer Microfluidizer Processor, Microfluidics, add 12.6 L of water for injection and disperse at 60 MPa (592 atm) at (40-45) oC subject to control of the liquid under homogenisation for optical density. Once optical density is 0.15 (wavelength is 540 nm; absorption layer thickness is 0.5 cm), add sterile solution of 480 g of lactose (milk sugar, pharmacopoeia grade) in 2.4 L of water for injection to the resulting emulsion. Continue dispersing in the reactor of the high pressure homogenizer until optical density is 0.15.

Filter the resulting emulsion sequentially using Millipore device, starting with 0.45 μm and 0.22 μm membranes, followed by sterilisation filtration and dispense into glass vials under aseptic conditions. Allow vials with the emulsion to freeze efficiently and freeze-dry in TG-50 device. After drying, blow vials containing a lyophilised product with inert gas, close and seal under aseptic conditions.

Reproduction of the prototype method allows obtaining a product in the form of a light amorphous substance having light-yellow colour with a touch of lemon and characteristic odour.

For identity testing and establishing the quality of target liposomal products, obtained by the method claimed and the prototype one, the products used have the form of emulsion reconstituted by adding a sterile isotonic 0.9% sodium chloride solution at 37° C. to vials with lyophilised product, this corresponds to the form of pharmacotherapeutic administration thereof.

In terms of the pharmaceutical quality of the target product, the efficacy of the method claimed was confirmed by the qualitative and quantitative identification of QC and a lipid component PC and liposomal status of the target product using a series of independent physical and chemical methods, specifically:
Spectrophotometry method based on parameters of characteristic absorption at wavelengths (255-259) nm and (373-377) nm and optical density at wavelength (375±2) nm of the target product solution in ethyl alcohol compared to those of quercetin standard solutions (QC identity and assay, respectively);
Brown-green colouring, when iron (III) chloride solution is added to the target product solution in ethyl alcohol (identity of QC phenol hydroxyl group);
Thin-layer chromatography method based on a chromatogram of the target product solution in ethyl alcohol, whereon there is a yellow spot produced by PC at the level of the main spot of standard PC (PC identity);
Spectrophotometry method based on optical density at wavelength (830±2) nm for products of the colour reaction of the target product in a mixture of ethyl alcohol and water treated with perchloric acid, and ammonium molybdate and amidol compared to that of standard PC (PC assay);
Gel-filtration of the emulsion reconstituted from lyophilised target product using a Sefadex G-25 column (effluent—isotonic 0.9% sodium chloride solution) subject to control for output of the liposomal product based on characteristic absorption at wavelength of 540 nm (liposome identity);
Measuring particle size in the liposomal product emulsion by dynamic light scattering (DLS) method;
Oxidation index of the liposomal fraction in the target product compared to that of the standard PC (liposome stability test);
Duration of production, emulsion breakdown stability and stability of dispersion structure of the emulsion reconstituted from the lyophilised target product (determination of the functional stability and liposome distribution by size);
pH values and osmolality of the emulsion (to determine whether the products meet requirements to functional application of parenteral and ophthalmologic products).

Based on physicochemical identity tests (Table 2), the method claimed ensures the quality and stability of the target product as QC-containing liposomes derived from phosphatidylcholine, specifically:
Based on spectroscopy, thin-layer chromatography and chemical analysis, the target product features the native composition and the nature of QC and PC corresponding to those of standard quercetin and phosphatidylcholine;
QC and PC, as components of the target product, are quantitatively included to liposomes based on gel-filtration method;
The target product is characterised by quick formation of an emulsion from lyophilisate and substantial resistance to emulsion breakdown;
In the emulsion of the target product, the stable liposome size of 174±2 nm is accompanied by practical monodispersity of distribution by size and low oxidation index;
The emulsion has stable pH value that meets physiological norms for this value in vivo;
Osmolality value meets pharmacopoeia requirements.

The target product features these characteristic factors in Examples 1-3 (Table 1), however implementation of the method claimed with other parameters (Examples 4-6) and as suggested by the prototype method (Example 7) leads to poorer quality and stability of the target product:
Larger size and inhomogeneous dispersion of liposomes;
Relative increase in the oxidation index of the liposomal product;
Emulsion takes longer time for reconstitution from lyophilised product increases while the time for emulsion breakdown reduces (by visual observation);
pH value of the emulsion is out of the range of physiologically acceptable values.

The advantages established in terms of quality and stability of the target product in Examples 1-3 (Table 1) correlate well with the time reduced and operations of the method claimed, simplified to certain extent, as compared with parameters of these operations in Examples 4-6 and the prototype method (Example 7):
A heating operation to dissolve QC is omitted;
After emulsification, time for mixing is now cut by 10-15 times;
The dispersion period is reduced by 1.5-2 times, and the average filtration time is also reduced.

Therefore, the method claimed in Examples 1-3 allows producing a target liposomal QC product which, based on its pharmaceutical quality, meets the requirements to parental products and, at the same time, provides certain technological benefits in terms of method reproduction.

According to the object of the invention, the quality of the target product—a QC-containing liposomal product—was assessed based on values of pharmacological activity in non-clinical studies in cardiologic and ophthalmologic pathology.

The design of studies corresponds to the object of the invention and the evidence-based studies were to prove pharmacological quality of the target product, when various routes of administration are used.

Specific pharmacological activity of target products produced by the method claimed (Examples 1-6) and the prototype method were compared in the following experimental models:
1) subtotal myocardial ischemia and reperfusion—assessment of how the emulsion of the lyophilised target product (0.2-0.6 mg/mL) impacts isolated heart performance in Guinea pigs by Langendorff method;
2) traumatic keratitis—assessment of how the emulsion of the target product in a physiological solution impacts cornea reparation in rabbits following instillation with 0.06 mL of the emulsion (containing 18.8 mg/mL of the lyophilised target product).

Pharmacological safety of target products produced by the method claimed and the prototype method was also assessed. In order to comply with GLP requirements and bioethical standards requiring non-clinical studies to use an optimised number of animals, the safety of target products was defined for a typical example of implementing the method claimed (Example 1, Table 1) and the prototype method (Example 7) using two routes of administration.

After single-dose intravenous administration of emulsion and following repeated intraperitoneal injections (14 days), the target products caused no deaths in test animals (white mice, white rats), no local irritative effects, negative impact on body weight and general behavioural reactions, are not associated with any changes in blood counts, serum biochemistry, signs of an inflammatory reaction and dystrophic changes in organ and tissue morphology. After repeated frequent intraocular instillations (every 15 min for 6 hours) and after daily four-time intraocular instillations for 28 days in rabbits, liposomal products caused no local irritative and allergising effects, negative impact on corneal epithelium continuity, eye rigidity and the state of external eye structures.

Therefore, in terms of parameters of acute and chronic toxicity, including ophthalmologic harmlessness, being identical for both target products, the products should be classified as almost harmless, which give grounds for pharmacotherapeutic use thereof.

Table 3 contains efficacy assessment of the method claimed based on the cardioprotective activity of the target product, and Table 4—based on dynamics of reparative and anti-inflammatory effect in an ophthalmologic experiment. In both pathology models used, all products improve the parameters of physiological condition, however the method implementation claimed ensures the product quality, which is associated with higher pharmacological effect.

In terms of impact on functional activity of heart in ischemia and reperfusion, the products feature:
higher antiarrhythmic effect demonstrated by dramatic reduction in the number of extrasystoles associated with both ischemia (product produced by the method claimed—by 53-59%; product by the prototype method—by 45%), and reperfusion (product by the method claimed—by 78%; product by the prototype method—by 44%);
more expressive protective effect in terms of the heart rate associated with ischemia and dynamics of heart rate normalisation after reperfusion;
normalizing effect on ischemia-associated hemodynamic characteristics of the myocardium and normalisation of these characteristics after reperfusion.

Notably, the product produced by the method claimed is competitive based on heart functional activity even when used in lower doses compared to the product produced by the prototype method.

In terms of impact on characteristic parameters in the model of traumatic keratitis, the products produced by the method claimed ensure:
activation and accelerated dynamics of healing in a traumatised eye (reparation of a deepithelised area);
substantial reduction of inflammation signs.

The results of comparison of pharmacological activity of target products in experimental pathologies having various origins proves both the high quality of liposomal quercetin product, as have been already confirmed by pharmaceutical and physicochemical findings, and its compliance with the desired polytropic pharmacotherapeutic effect.

The highest integral quality is inherent to products produced by the method claimed in line with parameters in Examples 1-3 and different from those of the prototype method, such as: quercetin is dissolved at a room temperature, the mixture is emulsified at (37-42) oC using lactose solution in phosphate buffer, pH (6.8-7.1), containing (70-90) % of total lactose, as an aqueous medium, the emulsion is dispersed subject to a stage-by-stage pressure increase from 300 atm to 1,200 atm and the emulsion is controlled for particle size, after dispersing, lactose solution in phosphate buffer, pH (6.8-7.1), containing (30-10) % of total lactose, is further added to the emulsion, and quercetin to lactose mass ratio is between (1:31) and (1:80). A deviation from these parameters (Examples 4-6 and the prototype method, Example 7) prolongs the method and makes it somewhat more difficult (Table 1), the high quality of the target product, identified as stable liposomal quercetin product, is not maintained, and, hence, higher pharmacological activity of the product is not ensured.

The optimal combination of favourable technological profiles of operations and processes and positive physicochemical identification and data on pharmacological effects and harmlessness of the target product demonstrates the benefits of the method claimed compared to the prototype one when the object is to obtain a stable pharmacologically active liposomal quercetin product. The product produced by the method claimed compares favourably with a QC-containing liposomal product produced by the prototype method.

These considerations prove that it is advisable to use the method claimed to produce an efficient liposomal medicinal product quercetin, having a wide pharmacotherapeutic profile suitable to various routes of administration.

TABLE 1
Parameters to produce liposomal QC-containing product
by the method claimed and the prototype method

| | Example No. | | | | | | |
|---|---|---|---|---|---|---|---|
| | | | | | | | 7 |
| | | | | | | | Proto- |
| | Method claimed | | | | | | type |
| | 1 | 2 | 3 | 4 | 5 | 6 | method |
| PC:QC ratio introduced | 1:0.030 | 1:0.020 | 1:0.060 | 1:0.030 | 1:0.050 | 1:0.060 | 1:0.030 |
| (mass fraction) | | | | | | | |
| QC dissolution | | | | | | | |
| temperature: | | | | | | | |
| Room | + | + | + | − | − | + | − |
| temperature | | | | | | | |
| 50° C. | − | − | − | + | + | − | + |
| Emulsification | | | | | | | |
| parameters: | | | | | | | |
| a) Medium: | | | | | | | |
| Water | − | − | − | + | − | − | + |
| Lactose solution in | + | + | + | − | + | + | − |
| buffer, pH (6.7-7.0) | | | | | | | |
| b) Temperature, ° C. | 40 | 42 | 37 | 36 | 44 | 25 | * |
| b) Mixing time, min. | 20 | 35 | 30 | 35 | 35 | 70 | 120 |
| c) Amount of lactose | 80 | 90 | 70 | 0 | 69 | 91 | 0 |
| introduced (% of total) | | | | | | | |
| Dispersion | | | | | | | |
| parameters: | | | | | | | |
| a) Pressure at cycles | | | | | | | |
| (atm): | | | | | | | |
| Cycle 1-3 | 300 | 300 | 300 | 600 for | 300 | 300 | ~600 for |
| Cycle 4-6 | 600 | 300 | 600 | entire | 800 | 400 | entire |
| Cycle 7-8 | 600 | 300 | 900 | process | 1000 | 600 | process |
| from Cycle 9 until | 900 | 900 | 1200 | | 1300 | 1000 | |
| the end of the process | | | | | | | |
| b) Particle size after | <180 | <180 | <180 | <260 | <180 | <250 | <300* |
| dispersion, nm | | | | | | | |
| c) Introduction of | | | | | | | |
| lactose solution: | | | | | | | |
| during dispersion | − | − | − | + | − | − | + |
| after dispersion | + | + | + | − | + | + | − |
| d) Amount of lactose | 20 | 10 | 30 | 100 | 31 | 9 | 100 |
| introduced (% of total) | | | | | | | |
| Average filtration | 20 | 18 | 30 | 45 | 30 | 30 | 48 |
| period after dispersion | | | | | | | |
| (per L), min | | | | | | | |
| QC:lactose ratio | 1:50 | 1:30 | 1:80 | 1:81 | 1:24 | 1:50 | 1:24 |
| (mass fraction) | | | | | | | |

(+) - the parameter was applied;
(−) - the parameter was not applied;
(*) - the parameter is not regulated by the prototype method.

TABLE 2
Parameters of the pharmaceutical quality of the target product
produced by the method claimed and the prototype method

| | Example No. | | | | | | |
|---|---|---|---|---|---|---|---|
| | | | | | | | 7 |
| | Method claimed | | | | | | Prototype |
| | 1 | 2 | 3 | 4 | 5 | 6 | method |
| QC identity | + | + | | | | | + |
| PC identity | + | + | | | | | + |
| PC:QC ratio in | | | | | | | |
| liposomes (mass | | | | | | | |
| fraction): | | | | | | | |
| before gel- | 1:0.030 | 1:0.020 | 1:0.050 | 1:0.030 | 1:0.050 | 1:0.050 | 1:0.030 |
| filtration | | | | | | | |
| after gel- | 1:0.030 | 1:0.020 | 1:0.050 | 1:0.026 | 1:0.048 | 1:0.049 | 1:0.028 |
| filtration | | | | | | | |
| QC inclusion in | 100 | 100 | 100 | 95 | 98 | 99 | 99 |
| liposomes, % (of | | | | | | | |
| total introduced) | | | | | | | |
| Liposome size | 173/95 | 176/100 | 175/94 | 193/70 | 185/60 | 180/85 | 280/72 |
| (nm)/content of | 60/5 | | 50/6 | 80/30 | 90/30 | 50/15 | 80/16 |
| liposomes of | | | | | 50/10 | | 50/12 |
| respective size | | | | | | | |
| (%) * | | | | | | | |
| Oxidation index | 0.25 | 0.23 | 0.25 | 0.31 | 0.30 | 0.27 | 0.30 |
| Emulsification | 1.7 | 1.2 | 1.5 | 1.9 | 2.1 | 1.8 | 1.9 |
| time, min. * | | | | | | | |
| Emulsion | 88 | 90 | 72 | 68 | 56 | 70 | 40 |
| breakdown | | | | | | | |
| stability, min. * | | | | | | | |
| pH value of | 6.7 | 6.6 | 6.8 | 6.0 | 6.2 | 6.0 | 5.3 |
| emulsion * | | | | | | | |
| Emulsion | 380 | 320 | 400 | 420 | 330 | 380 | 328 |
| osmolality, | | | | | | | |
| mosmol/g ** | | | | | | | |

(+) - positive identification;
* defined for emulsion originally reconstituted from a lyophilised product;
** According to pharmacopoeia requirements, osmolality must be within 214-434 mosmol/g for ophthalmologic products.

TABLE 3
Efficacy of the method claimed vs. the prototype method based on the pharmacological
quality of liposomal target product in ischemia and perfusion in isolated heart

| | Example No. (as per Table 1) | | | | | | |
|---|---|---|---|---|---|---|---|
| | | | | | | | 7 |
| Pharmacological | Method claimed | | | | | | Prototype |
| quality parameters | 1* | 2** | 3* | 4* | 5** | 6* | method** |
| Antiarrhythmic | | | | | | | |
| effect (number of | | | | | | | |
| extrasystoles/min): | | | | | | | |
| Ischemia | 7.5 | 6.7 | 7.8 | 8.5 | 7.8 | 8.5 | 9.0 |
| (control 16.50)*** | | | | | | | |
| Reperfusion (control | 4.0 | 4.0 | 4.2 | 6.2 | 6.4 | 8.6 | 10.5 |
| 18.83)*** | | | | | | | |
| Changes in heart | | | | | | | |
| rate, bpm | | | | | | | |
| (±5) in duration: | | | | | | | |
| Ischemia (min): | | | | | | | |
| 0 | 150 | 165 | 160 | 155 | 169 | 150 | 158 |
| 40 | 61 | 76 | 60 | 50 | 51 | 65 | 51 |
| Reperfusion (min) | | | | | | | |
| 5 | 170 | 170 | 165 | 150 | 153 | 145 | 177 |
| 60 | 160 | 165 | 158 | 160 | 160 | 145 | 190 |
| Changes in pressure | | | | | | | |
| in the left ventricle | | | | | | | |
| (% of baseline) | | | | | | | |
| (±5) in duration: | | | | | | | |
| Ischemia (min): | | | | | | | |
| 0 | 100 | 100 | 100 | 100 | 100 | 100 | 100 |
| 40 | 25 | 30 | 38 | 22 | 30 | 20 | 20 |
| Reperfusion (min) | | | | | | | |
| 5 | 90 | 90 | 85 | 70 | 78 | 80 | 82 |
| 60 | 90 | 95 | 95 | 75 | 80 | 75 | 78 |
| Changes in pressure | | | | | | | |
| in coronary vessels | | | | | | | |
| (mm Hg) in duration: | | | | | | | |
| Ischemia (min): | | | | | | | |
| 0 | 68 | 70 | 70 | 72 | 70 | 65 | 72 |
| 40 | 15 | 15 | 20 | 10 | 12 | 10 | 10 |
| Reperfusion (min) | | | | | | | |
| 5 | 69 | 65 | 60 | 70 | 72 | 70 | 74 |
| 60 | 66 | 70 | 68 | 81 | 80 | 78 | 80 |

*product emulsion concentration in experimental medium - 0.2 mg/mL;
**product emulsion concentration in experimental medium - 0.6 mg/mL;
***control - no product is used;
Conditions of the experiment: subtotal ischemia (90% of perfusion restriction) followed by reperfusion (90%); experimental medium - Krebs solution, animals - Guinea pigs, body weight 400-450 g, 10 animals per arm.

TABLE 4
Efficacy of the method claimed vs. the prototype method based on the pharmacological
quality of liposomal target product in experimental traumatic keratitis

| | Dynamics of pharmacological quality parameters (activity) * | | | | | |
|---|---|---|---|---|---|---|
| | Eye reparation: | | | | | |
| Study object - | deepithelised area, | | | Eye inflammatory reaction | | |
| product as per | mm² (±10-±1.8) | | | (total score**) | | |
| Example | Day 2 | Day 5 | Day 8 | Day 2 | Day 5 | Day 8 |

Products by the method claimed under examples:

| | | | | | | |
|---|---|---|---|---|---|---|
| 1 | 46.5 | 6.9 | 1.3 | 2.8 | 2.3 | 1.4 |
| 2 | 47.0 | 7.1 | 1.7 | 3.8 | 2.6 | 1.5 |
| 3 | 45.0 | 5.7 | 0 | 2.1 | 2.1 | 0 |
| 4 | 48.0 | 7.8 | 2.0 | 5.0 | 2.8 | 1.6 |
| 5 | 48.3 | 7.4 | 1.8 | 5.2 | 3.0 | 1.9 |
| 6 | 47.9 | 7.0 | 2.0 | 4.9 | 2.8 | 1.8 |

Products by the prototype method claimed under example:

| | | | | | | |
|---|---|---|---|---|---|---|
| 7 | 46.8 | 7.8 | 2.1 | 4.9 | 2.8 | 1.9 |
| Control*** | 56.9 | 40.1 | 12.4 | 12.0 | 20.2 | 8.4 |

* 8 eyes assessed per experimental arm;
** The parameter meets total inflammatory reaction in structures of anterior area of eye;
*** Control group with pathology, where instillations with physiological solutions were used.

1. The method of obtaining a pharmacologically active liposomal quercetin-containing product by producing a mixture of ethanol solutions of quercetin and phosphatidylcholine, drying the mixture in vacuum, emulsifying the mixture in an aqueous medium, dispersing the emulsion, stage-by-stage filtrating, sterilisation filtrating and freeze-drying, characterized in that quercetin is dissolved at a room temperature; the mixture is emulsified at (37-42) oC and lactose solution in phosphate buffer, pH (6.8-7.1), containing (70-90) % of total lactose, is used as an aqueous medium; dispersing is subject to a stage-by-stage pressure increase from 300 atm to 1,200 atm and the emulsion is controlled for particle size; after dispersing, lactose solution in phosphate buffer, pH (6.8-7.1), containing (30-10) % of total lactose, is further added to the emulsion, and quercetin to lactose mass ratio is between (1:31) and (1:80).
